(19) United States
(12) Patent Application Publication
(10) Pub. No.: US 2021/0016034 A1
(43) Pub. Date: Jan. 21, 2021

(54) Apparatus for Adjusting the Tidal Volume Delivered by a Resuscitation Bag and Methods for Using the Same

(71) Applicants: Steve Islava, Newport Beach, CA (US); John Guy Cazort, III, Corona del Mar, CA (US); Richard A. Van Auken, Aliso Viejo, CA (US)

(72) Inventors: Steve Islava, Newport Beach, CA (US); John Guy Cazort, III, Corona del Mar, CA (US); Richard A. Van Auken, Aliso Viejo, CA (US)

(21) Appl. No.: 16/927,443

(22) Filed: Jul. 13, 2020

Related U.S. Application Data
(60) Provisional application No. 62/875,166, filed on Jul. 17, 2019.

(51) Int. Cl. A61M 16/00

(52) U.S. Cl. CPC A61M 16/0084; A61M 2205/6081; A61M 2202/0208

(57) ABSTRACT

An apparatus and method for selecting a tidal breath to be delivered to a patient with a resuscitation bag according to the patient's size or age. A user estimates a proper tidal breath for a patient and then adjusts or fixes the volume of a resuscitation bag to deliver that selected tidal breath. The user may deploy a variety of mechanical means to either limit the effective volume of the resuscitation bag or temporary maintain the resuscitation bag in a deformed configuration which also restricts or reduces the internal volume of the resuscitation bag. The mechanical means for limiting the volume of the resuscitation bag is also marked so as to notify the user the current degree by which the volume of the resuscitation bag is reduced, thereby informing the user the size of the tidal breath that is delivered when the resuscitation bag is compressed.

### RELATED APPLICATIONS

This application claims priority to, and the benefit of the earlier filing date of US provisional patent application entitled “Apparatus for Adjusting the Tidal Volume Delivered by a Resuscitation Bag and Methods for Using the Same,” filed on Jul. 17, 2019, Ser. No. 62/875,166, pursuant to 35 USC 120, the contents of all of which are incorporated herein by reference.

### BACKGROUND

### Field of the Technology

The invention relates to the field of emergency medical devices, particularly to mobile resuscitation devices.

### Description of the Prior Art

Resuscitation devices have long been used by emergency response personnel and other medical professionals to forcibly send air or oxygen into the lungs of an injured or unconscious patient. Many of these devices take the form of a resuscitation bag that is connected to a face mask which is placed over the patient's nose and mouth. The resuscitation bag may also be attached to an endotracheal tube, or other airway device. The bag also typically comprises a one-way directional valve which is in turn coupled to a reservoir, an oxygen source, or is instead open to the ambient environment. After the face mask has been properly placed, the EMT or medical professional squeezes or compresses the bag which forces air contained within the bag to travel through the face mask and enter the patient's oral airway. When the medical professional releases the bag or otherwise relaxes their grip, air from the environment or the reservoir enters the bag through the one-way directional valve to fill the vacuum created within the bag. The medical professional continues to ventilate the patient by repeatedly squeezing the bag and delivering air to the patient's lungs for as long as their specific treatment requires.

However, as has become increasingly accepted within the medical community, providing tidal volumes to a patient which are too large or at too high a pressure can be harmful to the patient and can in fact produce lung injury. For example, over inflating a child or adult causes air to enter the stomach or may rupture the alveoli. Additionally, it is very easy to over inflate a patient in an emergency situation when the added stress and surrounding circumstances of the emergency can cause unintentional exuberance on the part of the treating medical professional. Previously, it was thought that 10 to 15 ml of air/oxygen per kilogram of the patient weight was thought to be sufficient, however recently it has become apparent that tidal volumes of only 6 to 8 ml of air/oxygen per kilogram of patient weight are ideal for avoiding further acute lung injury in the patient.

It is therefore critical for a patient requiring ventilation that the resuscitation device provides a tidal volume which matches the patient's specific lung size. In other words, the resuscitation device must consistently and reliably deliver a volume of air/oxygen which is large enough to provide the patient sufficient ventilation but not so large that the patient's lungs are injured. This can be difficult however using a resuscitation bag since the volume of air which is delivered to the patient is dictated by the amount or how hard the medical professional squeezes the bag. The medical professional using a resuscitation bag must therefore estimate the proper amount of air/oxygen by observing the rise and fall of the chest as well as resistance caused by the lungs filling. After estimating a proper amount, the EMT must then actually compress the bag to the required degree in order to deliver that desired tidal volume each and every time, a process which the medical professional has no ability to verify since many resuscitation devices do not have a means for measuring or displaying the volume of air which is squeezed from the bag. Because the specific volume of air can vary significantly each time the medical professional compresses the bag, it is almost impossible for the medical professional to deliver a consistent tidal volume to the patient which is correctly keyed to their specific lung capacity.

Several resuscitation devices exist which comprise several different sized bags, wherein each bag is configured to deliver a different tidal volume when compressed. For example, the resuscitation device comprises a face mask which may be detached and reattached to an appropriately sized removable bag, namely a large bag for adults and a smaller bag for smaller adults or children. This type of device can be problematic however for EMTs and other medical professionals who work in confined areas such as ambulances and field hospitals where storage space is at a premium thus making the storage of multiple resuscitation bags inconvenient if not impractical. Furthermore, EMTs and other trained medical professionals, who are often operating under emergency conditions need a fast, reliable way to deliver the correct tidal volume with, a minimum of extraneous effort so that the patient can be treated properly as soon as possible.

Further devices include specialized ventilators which may be adjusted to deliver patient specific tidal volumes, however these devices are standalone items, thereby requiring the EMT or other medical personnel to purchase brand new equipment instead of repurposing or retrofitting a preexisting resuscitation device which already may be in the EMT's inventory. Additionally, a ventilator may not be the most practice piece of medical equipment for EMTs or other mobile medical professionals because of their relative size, weight, and power consumption requirements.

What is needed therefore is a means for quickly and accurately adjusting the volume of air delivered to a patient from a resuscitation bag and a method for using the same. The adjustment means may be employed in a means-specific resuscitation bag or, alternatively, may be a retro fit device disposed on or inserted into a pre-existing means for resuscitation.

### BRIEF SUMMARY

The current invention provides an apparatus for providing a selected tidal breath to a patient. The apparatus includes a resuscitation bag comprising a first volume, a mechanical means for reducing the first volume of the resuscitation bag to obtain a second volume, and a mechanical means for maintaining the second volume of the resuscitation bag even during a series of compressions of the resuscitation bag. It is critical to note that the second volume of the resuscitation bag corresponds to the selected tidal breath which is subsequently delivered or sent to the patient.

In one embodiment, the specific mechanical means for reducing the first volume of the resuscitation bag to obtain the second volume includes an inflatable core which is disposed within the resuscitation bag and a hand pump which is in turn connected to the inflatable core. Additionally in this embodiment, the means for maintaining the second volume of the resuscitation bag during a series of compressions of the resuscitation bag includes the inflatable core being comprised of a rigid, non-deformable material.

In another embodiment, the mechanical means for reducing the first volume of the resuscitation bag to obtain the second volume includes an adjustable cage which is disposed within the resuscitation bag and a central arm that is inserted through an end of the resuscitation bag and connected to the adjustable cage. Here, the second volume of the resuscitation bag is maintained during a series of compressions via a plurality of rigid hinges disposed in a corresponding plurality of spokes of the adjustable cage.

In yet another embodiment, the mechanical means for reducing the first volume of the resuscitation bag to obtain the second volume involves a removable strap that is disposed or wrapped around an outer circumference of the resuscitation bag. The removable strap itself includes a segment of coupling material disposed on the strap in order to maintain the second volume of the resuscitation bag during a series of compressions.

In yet another embodiment, the mechanical means for reducing the first volume of the resuscitation bag to obtain the second volume includes a drawstring that is disposed through the first volume of the resuscitation bag and along a lateral axis of the resuscitation bag. Here, the drawstring is also threaded through a spring stop disposed on an outside surface of the resuscitation bag in order to maintain the second volume of the resuscitation bag during a series of compressions of the resuscitation bag.

In a further embodiment, the mechanical means for reducing the first volume of the resuscitation bag to obtain the second volume includes an adjustable wire that is disposed between at least two lateral surfaces of the resuscitation bag, wherein at least a segment of the adjustable wire is specifically disposed across a portion of an outside surface of the resuscitation bag. Here, a pair of opposing ends of the adjustable wire are connected to opposing sides of a ratchet. The ratchet itself is disposed on the outside surface of the resuscitation bag at a position which opposes the position of the segment of adjustable wire that is disposed on the outside surface of the resuscitation bag.

In a separate embodiment, the mechanical means for reducing the first volume of the resuscitation bag to obtain the second volume includes a plurality of ribs that are embedded into an outer surface of the resuscitation bag. Additionally, a telescoping rod is disposed through a longitudinal axis of the resuscitation bag. Connected to both the plurality of ribs and the telescoping rod is an adjustable dial which helps maintain the second volume of the resuscitation bag during a series of compressions of the resuscitation bag.

In yet another embodiment, the mechanical means for reducing the first volume of the resuscitation bag to obtain the second volume includes an upper plate and a lower plate, where the lower plate is disposed on an opposing surface of the resuscitation bag relative to the upper plate. An adjustment pin configured to hold the lower plate at a fixed position relative to the upper plate is also included in order to maintain the second volume of the resuscitation bag during a series of compressions of the resuscitation bag.

The invention also provides a method for providing a selected tidal breath to a patient. The method includes providing a resuscitation bag with a first volume of air or oxygen, selectively altering the first volume of air or oxygen within the resuscitation bag to obtain a second volume of air or oxygen within the resuscitation bag, and then compressing the resuscitation bag to expel the second volume of air or oxygen from the resuscitation bag. Next, the second volume of the resuscitation bag is refilled with a fresh or new supply of air or oxygen.

In one embodiment, selectively altering the first volume of air or oxygen within the resuscitation bag to obtain a second volume of air or oxygen within the resuscitation bag includes inflating an inflatable core that is disposed within the first volume of air or oxygen which effectively reduces the first volume of air or oxygen to the second volume of air or oxygen.

In another embodiment, selectively altering the first volume of air or oxygen within the resuscitation bag to obtain a second volume of air or oxygen within the resuscitation bag involves expanding a cage that is disposed within the first volume of air or oxygen within the resuscitation bag which restricts the maximum amount the resuscitation bag may be compressed to the second volume of air or oxygen.

In yet another embodiment, selectively altering the first volume of air or oxygen within the resuscitation bag to obtain a second volume of air or oxygen within the resuscitation bag is performed by tightening a removable strap that is disposed or wrapped around the resuscitation bag so as to deform an outer surface of the resuscitation bag until the second volume of air or oxygen is obtained.

In a separate embodiment, selectively altering the first volume of air or oxygen within the resuscitation bag to obtain a second volume of air or oxygen within the resuscitation bag is performed by pulling a drawstring that is disposed through a lateral axis of the resuscitation bag until an outer surface of the resuscitation bag is deformed until the second volume of air or oxygen is obtained.

In one embodiment, selectively altering the first volume of air or oxygen within the resuscitation bag to obtain a second volume of air or oxygen within the resuscitation bag involves actuating a ratchet that is connected or disposed to a first lateral side of the resuscitation bag. Actuating the ratchet contracts a wire that is connected to a second lateral side of the resuscitation bag which in turn deforms an outer surface of the resuscitation bag until the second volume of air or oxygen is obtained. Here, the second lateral side of the resuscitation bag is disposed on an opposing or opposite side of the resuscitation bag relative to the first lateral side.

In yet a further embodiment, selectively altering the first volume of air or oxygen within the resuscitation bag to obtain a second volume of air or oxygen within the resuscitation bag is performed by actuating a dial which in turn compresses a plurality of ribs embedded in an outer surface of the resuscitation bag that are connected to the dial. Compressing the plurality of ribs deforms the outer surface of the resuscitation bag until the second volume of air or oxygen is obtained.

In another embodiment, selectively altering the first volume of air or oxygen within the resuscitation bag to obtain a second volume of air or oxygen within the resuscitation bag includes compressing the resuscitation bag between an upper plate and a lower plate until the second volume of air or oxygen is obtained and then locking a position of the lower plate relative to the upper plate.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents, under 35 USC 112. The disclosure can be better visualized by turning now to the following drawings wherein like elements are referenced by like numerals.

### BRIEF DESCRIPTION OF THE DRAWINGS

FIG. 1A is a side view of a first embodiment of the current invention comprising a hand pump and an inflatable core, the inflatable core disposed inside of a resuscitation bag and comprising a minimal inflated volume.

FIG. 1B is a side view of the embodiment seen in FIG. 1A when the inflatable core has been inflated to an intermediate volume.

FIG. 1C is a side view of the embodiment seen in FIG. 1A when the inflatable core has been inflated to a maximum volume.

FIG. 2A is a perspective view of a second embodiment of the current invention comprising a cage disposed in a resuscitation bag, the cage being disposed in a contracted configuration.

FIG. 2B is a side view of the second embodiment of the current invention seen in FIG. 2A.

FIG. 3A is a perspective view of the second embodiment of the current invention seen in FIG. 2A, the cage being disposed in a partially expanded configuration.

FIG. 3B is a side view of the second embodiment of the current invention seen in FIG. 3A.

FIG. 4A is a perspective view of the second embodiment of the current invention seen in FIG. 2A, the cage being disposed in a fully expanded configuration.

FIG. 4B is a side view of the second embodiment of the current invention seen in FIG. 4A.

FIG. 5 is a side view of a third embodiment of the current invention comprising a removable strap disposed around the volume of a resuscitation bag.

FIG. 6A is a perspective view of a fourth embodiment of the current invention comprising a drawstring disposed through a resuscitation bag. A user is also seen gripping a distal end of the drawstring while also manipulating a spring stop coupled to the drawstring.

FIG. 6B is a perspective view of the embodiment seen in FIG. 6A after the user has pulled the drawstring until a first marker disposed on the drawstring has reached the spring stop.

FIG. 6C is a perspective view of the embodiment seen in FIG. 6B as the user squeezes the resuscitation bag to apply a compression stroke after pulling the drawstring through the spring stop until the first marker is reached.

FIG. 6D is a perspective view of the embodiment seen in FIG. 6A after the user has pulled the drawstring until a second marker disposed on the drawstring has reached the spring stop.

FIG. 6E is a perspective view of the embodiment seen in FIG. 6D after the user has pulled the drawstring until a third marker disposed on the drawstring has reached the spring stop.

FIG. 6F is a perspective view of the embodiment seen in FIG. 6E as the user squeezes the resuscitation bag to apply a compression stroke after pulling the drawstring through the spring stop until the third marker is reached.

FIG. 6G is a perspective view of the embodiment seen in FIG. 6E after the user has pulled the drawstring until a fourth marker disposed on the drawstring has reached the spring stop.

FIG. 7A is a side cross sectional view of another embodiment of the current invention comprising a wire disposed through an internal volume of a resuscitation bag and coupled to an adjustable ratchet disposed on an outer surface of the resuscitation bag.

FIG. 7B is a perspective view of the embodiment seen in FIG. 7A.

FIG. 8A is a side cross sectional view of the embodiment seen in FIG. 7A after the ratchet has been actuated to compress and partially deform the resuscitation bag.

FIG. 8B is a perspective view of the embodiment seen in FIG. 8A.

FIG. 9A is a side cross sectional view of the embodiment seen in FIG. 7A after the ratchet has been actuated to compress and deform the resuscitation bag to a maximum degree.

FIG. 9B is a perspective view of the embodiment seen in FIG. 9A.

FIG. 10A is a perspective view of a user manipulating the embodiment of the current invention seen in FIGS. 8A and 8B by squeezing the resuscitation bag to apply a compression stroke after adjusting the ratchet to partially deform the resuscitation bag.

FIG. 10B is a perspective view of a user manipulating the embodiment seen in FIG. 10A by further adjusting the ratchet to further deform the resuscitation bag.

FIG. 10C is a perspective view of a user manipulating the embodiment of the current invention seen in FIG. 10B by squeezing the resuscitation bag to apply a compression stroke after adjusting the ratchet to further deform the resuscitation bag.

FIG. 10D is a perspective view of a user manipulating the embodiment seen in FIG. 10B by further adjusting the ratchet to further deform the resuscitation bag.

FIG. 10E is a perspective view of a user manipulating the embodiment of the current invention seen in FIG. 10D by squeezing the resuscitation bag to apply a compression stroke after adjusting the ratchet to further deform the resuscitation bag.

FIG. 10F is a perspective view of a user manipulating the embodiment seen in FIG. 10D by further adjusting the ratchet to deform the resuscitation bag to a maximum amount.

FIG. 10G is a perspective view of a user manipulating the embodiment of the current invention seen in FIG. 10F by squeezing the resuscitation bag to apply a compression stroke after adjusting the ratchet to deform the resuscitation bag to a maximum amount.

FIG. 11A is a side view of an alternative embodiment of the current invention comprising a dial and a telescoping rod disposed through the longitudinal axis of a resuscitation bag. The embodiment further includes a plurality of ribs which are spiraled around the surface and coupled to the dial so that when the dial is rotated, the surface of the resuscitation bag is deformed.

FIG. 11B is an end view of the alternative embodiment of the current invention seen in FIG. 11A.

FIG. 12A is a perspective view of an alternative embodiment of the current invention comprising a clamp disposed around and configured to interact with a resuscitation bag.

FIG. 12B is a side view of the alternative embodiment seen in FIG. 12A which in particular shows the upper plate, the lower plate, and rails which form the clamp.

FIG. 12C is an end view of the alternative embodiment seen in FIG. 12A.

FIG. 12D is a magnified view of the alternative embodiment seen in FIG. 12B and shows how the adjustable lower plate is coupled to the rails through a corresponding pair of apertures of the lower plate.

FIG. 12E is a magnified view of the alternative embodiment seen in FIG. 12V and shows how the adjustable lower plate is coupled to the rails through a corresponding pair of apertures of the lower plate.

FIG. 13A is a perspective view of the alternative embodiment seen in FIG. 12A after the clamp has been actuated and the resuscitation bag deformed.

FIG. 13B is a side view of the alternative embodiment seen in FIG. 13A.

FIG. 13C is an end view of the alternative embodiment seen in FIG. 13A.

The disclosure and its various embodiments can now be better understood by turning to the following detailed description of the preferred embodiments which are presented as illustrated examples of the embodiments defined in the claims. It is expressly understood that the embodiments as defined by the claims may be broader than the illustrated embodiments described below.

### DETAILED DESCRIPTION OF THE PREFERRED EMBODIMENTS

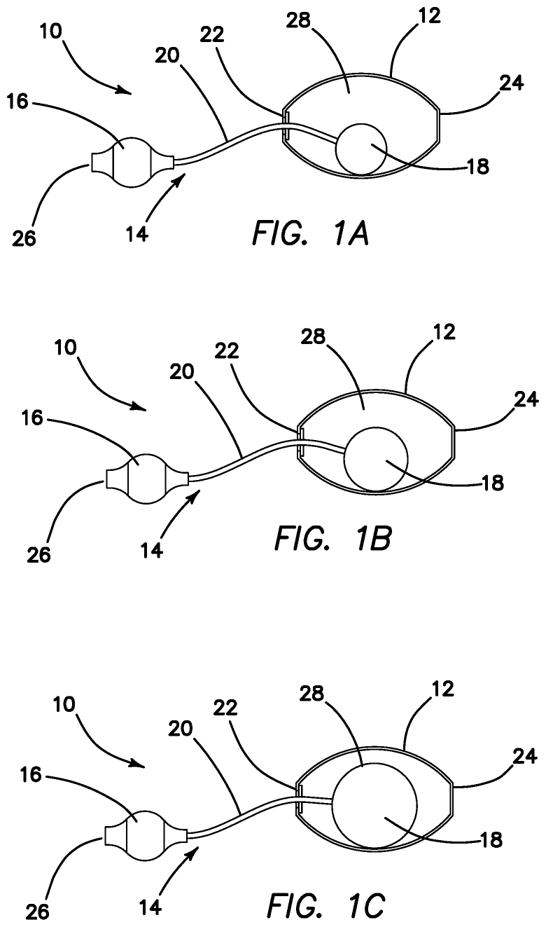

A first embodiment of the current invention may be seen by turning to FIGS. 1A-1C where the apparatus is denoted generally by reference numeral 10. Here, the apparatus 10 comprises a resuscitation bag 12 and an inflatable core 14. The inflatable core 14 itself comprises a hand pump 16 connected to an inflatable volume or core 18 via a flexible tube 20. The resuscitation bag 12 may be a resuscitation bag as is known in the art, namely wherein the resuscitation bag 12 is substantially oval or ovoid in shape and is comprised of a soft, yet durable material such as silicone, plastic, rubber, or any other suitable material now known or later devised. The resuscitation bag 12 further comprises an aperture at either lateral end, namely an inlet 22 and an outlet 24. The outlet 24 may accommodate a valve which in turn may be coupled to a face mask or other means for delivering air to a patient (not shown). The valve itself comprises an inlet, an outlet, and appropriate valving to allow the flow of air out from the internal volume of the resuscitation bag 12 when the bag 12 is compressed, and to allow externally provided air (i.e. from an oxygen source and/or the ambient environment) to enter the resuscitation bag 12 when the bag 12 is released as is well known in the art. The inlet 22 accommodates the flexible tube 20 in an air-tight fit or seal so as to prevent the air contained within the resuscitation bag 12 from escaping.

To use the apparatus 10, a user approaches a patient requiring ventilation and makes an estimate as to their age and/or weight. The user then actuates the hand pump 16 so as to inflate the inflatable core 18 which is already initially disposed within the internal volume 28 of the resuscitation bag 12. After each compression of the hand pump 16, ambient air/oxygen is drawn into the hand pump 16 through an aperture 26. The air/oxygen is then pumped through the flexible tube 20 and into the inflatable core 18. According to a predetermined measurement, the user actuates the hand pump 16 a defined number of times which in turn inflates the inflatable volume 18 to a predetermined degree. For example, if the patient is a large adult, the user may squeeze or compress the hand pump 16 for example five times thereby inflating the inflatable core 18 to the size seen in FIG. 1A. If the patient is a smaller adult or child, the user instead actuates the hand pump 16 until the inflatable core 18 is as big as seen in FIG. 1B. If the patient is an infant or exceptionally small, the user may selectively actuate the hand pump 16 until the inflatable core 18 is at a maximum size as seen in FIG. 1C. The resuscitation bag 12 is comprised of a material which is sufficiently transparent enough so that the user may visually judge the current size of the inflatable volume 18 and determine if it is the appropriate size for the patient being ventilated.

After the inflatable core 18 has reached the appropriate size and the face mask has been appropriately placed, the resuscitation bag 12 is ready for use by applying a number compressions followed by a corresponding number of release intakes. To perform a compression, the user squeezes or compresses the flexible surface of the resuscitation bag 12 which pushes the air/oxygen disposed in the volume 28 between the external surface of the inflatable core 18 and the internal surface of the resuscitation bag 12 out, through the outlet 24 and then subsequently to the patient. After a compression has been completed, the user then relaxes their grip on the resuscitation bag 12 which begins an intake stroke. After each time the resuscitation bag 18 is released, the surface of the bag 12 is resilient enough so that the bag 12 returns to its original shape which draws in new air/oxygen through the valving disposed in the outlet 24 and once again fills the volume 28 defined between the internal surface of the bag 12 and the external surface of the inflatable core 18. Because the inflatable core 18 in effect reduces the internal volume 28 of the resuscitation bag 12, the larger the inflatable core 18, the less air/oxygen there will be which is capable of being delivered to the patient through compression of the resuscitation bag 12. Conversely, if the inflatable core 18 remains small, the larger the volume of air the resuscitation bag 12 will expel with each compression. The material comprising the inflatable core 18 is sufficiently resilient or rigid so that even if the user over squeezes the resuscitation bag 12 and inadvertently presses against the inflatable core 18, the inflatable core 18 will not deform and therefore will not affect the volume of air/oxygen being delivered to the patient. The hand pump 16 further comprises a release valve or other aperture which allows the user to lower the air pressure within the inflatable core 18 which in turn decreases its overall size if the user determines that it is too large for the specific patient being treated. While the inflatable core 18 is shown in FIGS. 1A-1C as a sphere or ball, it is to be expressly noted that the inflatable core 18 may comprise any shape which effectively limits or restricts the internal volume of the resuscitation bag 12.

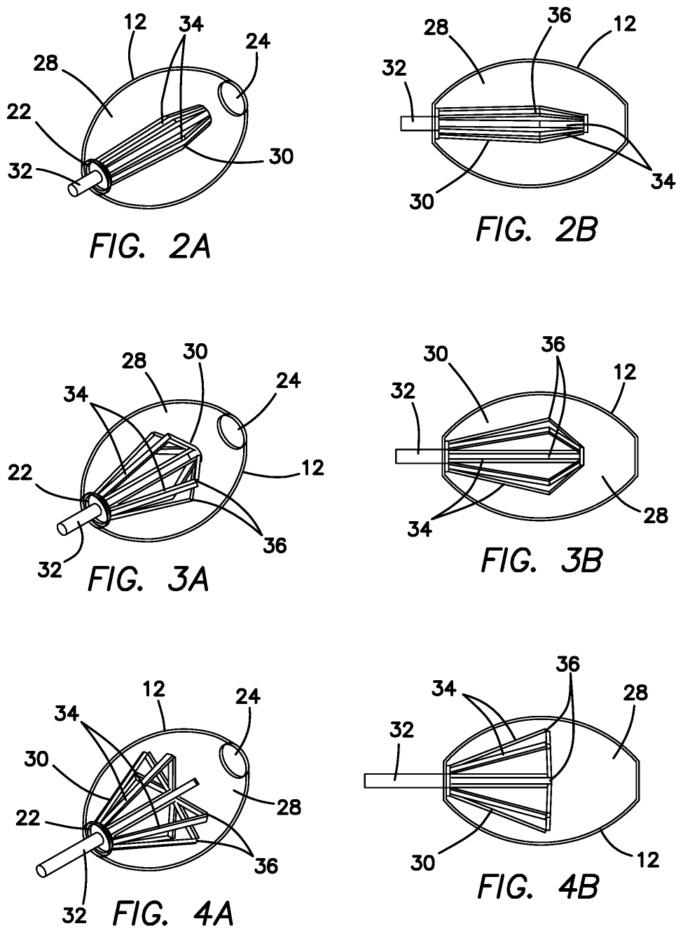

An alternative embodiment which incorporates a similar method of use may be seen in FIGS. 2A-4B. Here, an expandable cage 30 is disposed within the internal volume 28 of the resuscitation bag 12. The cage 30 is coupled to a central arm 32 which is disposed through and forms an air-tight seal with the inlet 22. The cage 30 is preferably inserted into the resuscitation bag 12 before the apparatus 10 is to be used, however the cage 30 may exist separately and then be inserted through the inlet 22, thereby retrofitting a resuscitation bag 12 or other means for resuscitation.

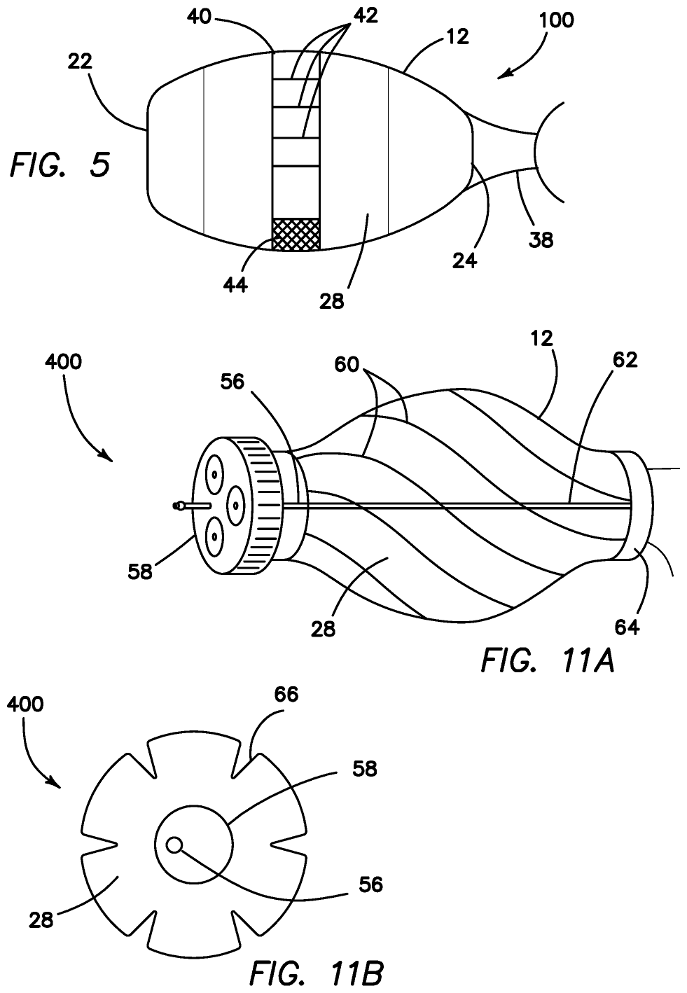

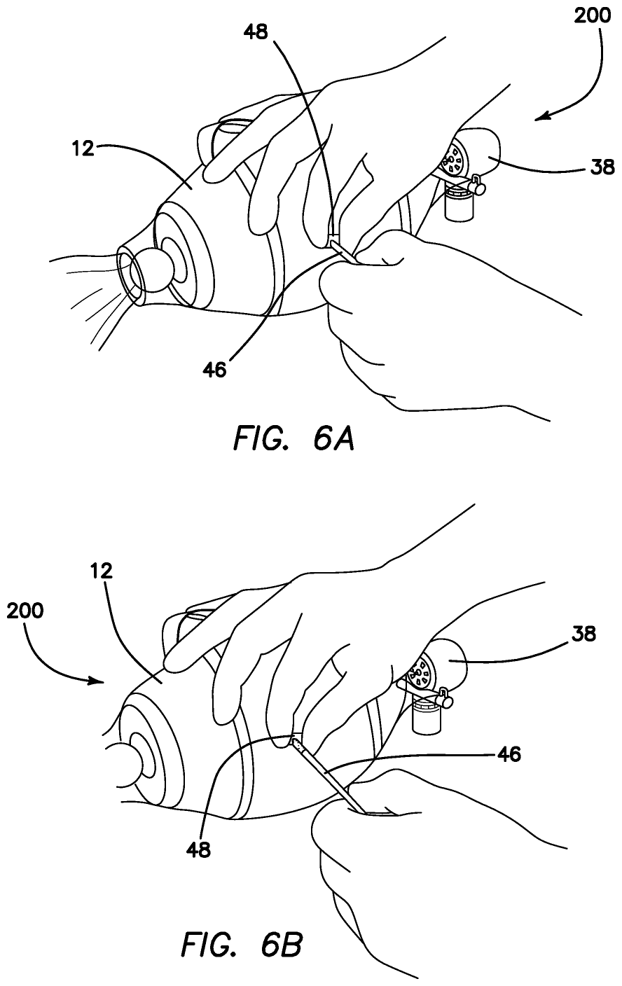

To use the alternative embodiment of apparatus 10, a user approaches a patient requiring ventilation and makes an estimate as to their age and/or weight. The user then actuates the central arm 32 so as to expand the cage 30 which is already disposed within the internal volume 28 of the resuscitation bag 12. The cage 30 may be expanded via the compression of a pump or the rotation of a wire disposed through the central arm 32, however the preferred method is to pull the central rod 32 in the proximal direction out through the inlet 22. As the central arm 32 is moved proximally through the inlet 22, a proximal portion of the cage 30 makes contact with the inside surface of the resuscitation bag 12 which begins to apply a force to a plurality of spokes 34 defining the cage 30. As the force applied to the spokes 34 increases, the spokes 34 begin to move outward and bend around a corresponding plurality of hinges 36. According to a known predetermined amount which is preferably denoted as a series of measurements or markings defined along the length of the central arm 32, the user continues to pull the central arm 32 in the proximal direction until the cage 30 has expanded to a desired degree. For example, if the patient is a large adult, the user pulls the central arm 32 to expand the spokes 34 of the cage 30 by rotating the spokes 34 about the hinges 36 until it is the size seen in FIGS. 2A and 2B. If the patient is a smaller adult or child, the user instead proximally pulls the central arm 32 until the spokes 34 are bent via the hinges 36 so that the cage 30 overall is as big as seen in FIGS. 3A and 3B. If the patient is an infant or exceptionally small, the user may selectively pull the central arm 32 until the spokes 34 of the cage 30 are bent about the hinges 36 at, a maximum amount as seen in FIGS. 4A and 4B with each spoke 34 comprising a substantially 90° bend therein. The resuscitation bag 12 is comprised of a material which is sufficiently transparent enough so that the user may visually judge the current size of the cage 30 and determine if it is the appropriate size for the patient being ventilated.

After the cage 30 has reached the appropriate size and the face mask has been appropriately placed, the resuscitation bag 12 is ready for use by applying a number compressions followed by a corresponding number of release intakes or intake strokes. To perform a compression, the user squeezes or compresses the flexible surface of the resuscitation bag 12 which pushes the air/oxygen disposed in the volume 28 between the outer contours of the cage 30 and the internal surface of the resuscitation bag 12 out through the outlet 24 and then subsequently to the patient. After a compression has been completed, the user then relaxes their grip on the resuscitation bag 12 which begins an intake stroke. After each time the resuscitation bag 18 is released, the surface of the bag 12 is resilient enough so that the bag 12 returns to its original shape which draws in new air/oxygen through the valving disposed in the outlet 24 and once again fills the volume 28 defined between the internal surface of the bag 12 and the cage 30. Because the cage 30 in effect reduces the internal volume 28 of the resuscitation bag 12, the more expanded the cage 30, the less air/oxygen there will be which is capable of being delivered to the patient through compression of the resuscitation bag 12. Conversely, if the cage 30 remains in a more contracted state as seen in FIG. 2B, the larger the volume of air the resuscitation bag 12 will expel with each compression. The hinges 36 of the cage 30 are sufficiently resilient or rigid so that even if the user over squeezes the resuscitation bag 12 and inadvertently presses against the spokes 34 of the cage 30, the hinges 36 will not bend or deform and therefore will not affect the volume of air/oxygen being delivered to the patient. To contract the cage 30 the user pushes the central arm 32 back in the distal direction which permits the spokes 34 to rotate about the hinges 36 and contract the cage 30 to a smaller overall shape if the user determines that it is too large for the specific patient being treated. While the cage 30 is substantially shown in FIGS. 2A-4B as a cone when fully expanded, it is to be expressly noted that the cage 30 may comprise any shape which effectively limits the internal volume of the resuscitation bag 12.

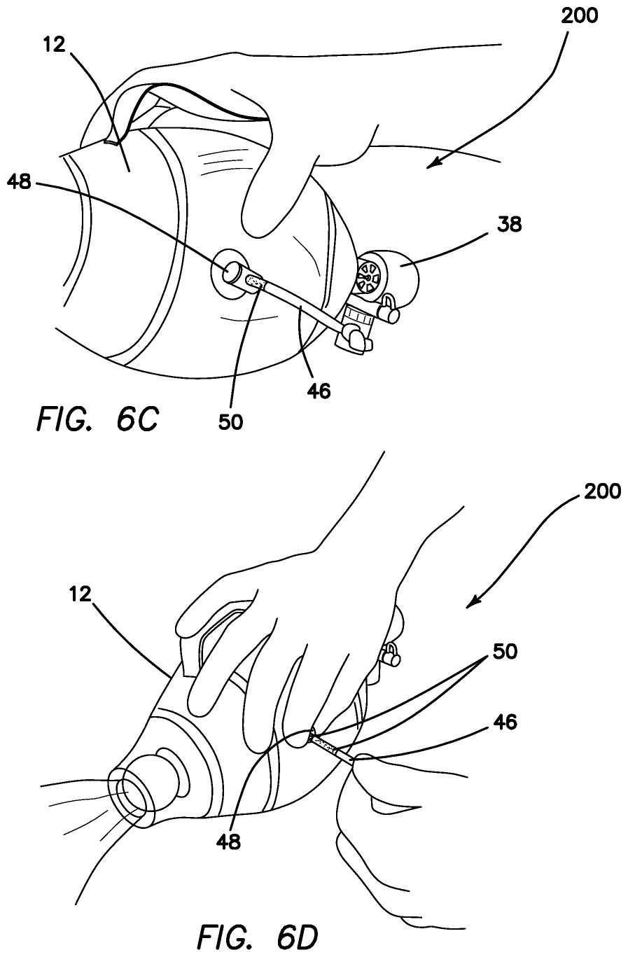

An alternative embodiment of the apparatus 100 may be seen by turning to FIG. 5. Here, the resuscitation bag 12 comprises a removable strap 40 which is wrapped or disposed around the outer circumference of the resuscitation bag 12. The strap 40 is preferably comprised of a strip of hook and loop fabric, specifically with one side of the strap 40 comprising a material consisting of a plurality of hooks while the reverse side comprises a corresponding material consisting of a plurality of loops which are configured to interact with the hook material. The strap 40 is applied to the resuscitation bag 12 by wrapping the strap 40 around the outer circumference of the bag 12 so that one end is brought around and layered or placed over the opposing end of the strap 40. Because the opposing surfaces of the strap 40 comprise complimentary coupling surfaces, the strap 40 is quickly and easily coupled to itself by simply pressing the opposing end of the strap 40 against the first end in order to engage the respective hook or loop fabric disposed thereon. In a related embodiment, the strap 40 may comprise a dedicated segment 44 of coupling material, such as hook and loop fabric, adhesive, or other material. The strap 40 is preferably coupled to the resuscitation bag 12 before treatment of the patient begins, however the strap 40 may also be initially detached and held separately until the user is ready to begin treatment.

To use the alternative embodiment of apparatus 100, a user approaches a patient requiring ventilation and makes an estimate as to their age and/or weight. The user then tightens the strap 40 which is already disposed around the external circumference of the internal volume 28 of the resuscitation bag 12. The user tightens the strap 40 by pulling the one end of the strap 40 free which detaches it from the fabric of the opposing end of the strap 40 disposed directly beneath it. The user then pulls the free end of the strap 40 in the proximal direction towards themselves which constricts the strap 40 about, the resuscitation bag 12 and begins to deform its surface, thus compressing the internal volume 28 within the resuscitation bag 12. Disposed or defined at a series of positions along the length of the strap 40 are a plurality of demarcations or markers 42. According to a known predetermined amount, each of the plurality of markers 42 correspond to a different tidal volume contained within the resuscitation bag 12 when the free end of the strap 40 is brought to that particular marker 42. Each marker 42 is denoted by a number, character, or is color coded so as to easily indicate to the user which marker 42 applies to which weight classification of the patient. The user continues to pull the strap 40 in the proximal direction until the resuscitation bag 12 has been compressed to a desired degree. For example, if the patient is a large adult, the user pulls the free end of the strap 40 to compress the resuscitation bag 12 until the free end is brought against the first marker 42. If the patient is a smaller adult or child, the user instead proximally pulls the free end of the strap 40 until the second or third marker 42 disposed on the strap 40. If the patient is an infant or exceptionally small, the user may selectively pull the free end of the strap 40 until reaching the fourth or final maker 42 disposed on the strap 40. At whatever marker 42 the free end of the strap 40 is pulled to, the strap 40 is then temporarily fixed at that position by the user coupling the strap 40 against itself, preferably through hook and loop fabric as discussed above or another quick adjust means.

After the strap 40 has been properly adjusted and the face mask has been appropriately placed, the resuscitation bag 12 is ready for use by applying a number compressions followed by a corresponding number of release intakes or intake strokes. To perform a compression, the user squeezes or compresses the flexible surface of the resuscitation bag 12 which pushes the air/oxygen disposed in the volume 28 out through the outlet 24 and then subsequently to the patient. After a compression has been completed, the user then relaxes their grip on the resuscitation bag 12 which begins an intake stroke. After each time the resuscitation bag 18 is released, the surface of the bag 12 is resilient enough so that the bag 12 returns to its user-adjusted shape which draws in new air/oxygen through the valving disposed in the outlet 24 and once again fills the volume 28. Because the strap 40 in effect reduces the amount that the internal volume 28 of the resuscitation bag 12 may be re-inflated, the tighter the strap 40 is about the resuscitation bag 12, the less air/oxygen there will be which is capable of being delivered to the patient through compression of the resuscitation bag 12. Conversely, if the strap 40 remains looser or in a more relaxed state, the larger the volume of air the resuscitation bag 12 will expel with each compression. The strap 40 is sufficiently tight or fixed into place around the resuscitation bag 12 so that even if the user completely releases the resuscitation bag 12, the strap 40 will not allow the resuscitation bag 12 to expand beyond its user-desired shape and therefore will not affect the volume of air/oxygen being delivered to the patient. To release the strap 40, the user pulls one end of the strap 40 back in the distal direction which separates the strap 40 from itself and loosens the strap 40 from the bag 12, allowing it to freely expand back to a new desired volume or even its original starting volume.

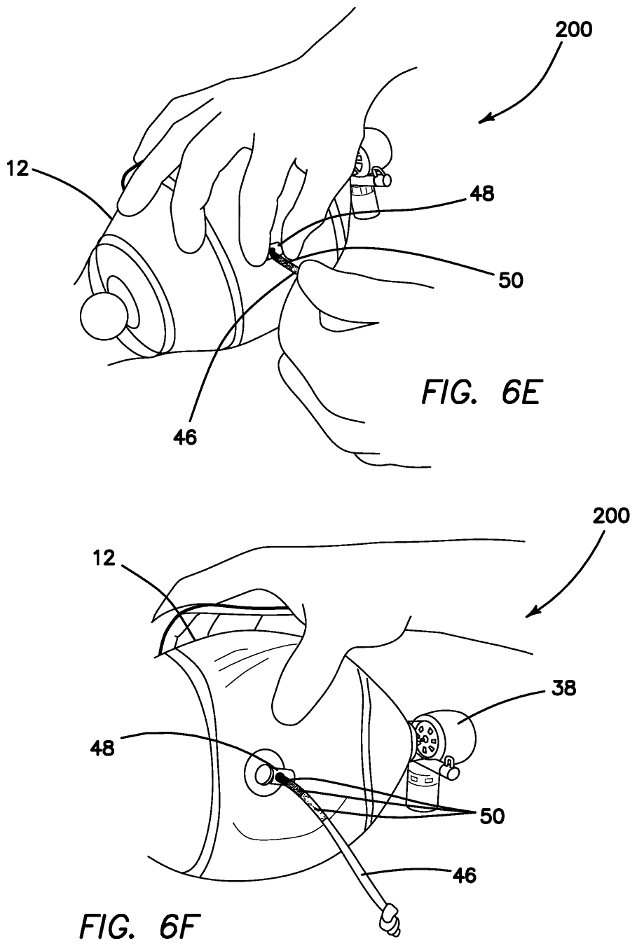

An alternative embodiment of the apparatus 200 may be seen by turning to FIGS. 6A-6G. Here, the resuscitation bag 12 comprises a drawstring 46 which is defined or disposed through the internal diameter of the resuscitation bag 12. The drawstring is preferably comprised of a rope or fabric, specifically with one end of the drawstring 46 anchored to a plug or stop disposed in one wall or surface of the resuscitation bag 12 with the remaining end disposed or threaded through a hole or aperture on the opposing surface or wall of the resuscitation bag 12. The drawstring 46 is also threaded through a cord stop or spring stop 48 which is disposed on the outside surface of the resuscitation bag 12.

To use the alternative embodiment of apparatus 200, a user approaches a patient requiring ventilation and makes an estimate as to their age and/or weight. The user then adjusts the drawstring 46 which is already disposed through the center of the internal volume 28 of the resuscitation bag 12. The user adjusts the drawstring 46 by actuating the spring stop 48 and then pulling the distal end of the drawstring 46 away from the outside surface of the resuscitation bag 12 as seen in FIGS. 6A and 6B. Because one end of the drawstring 46 remains stationary or fixed to the opposing inner surface of the resuscitation bag 12, pulling the distal end of the drawstring 46 while pushing proximally on the spring stop 48 deforms the surface of the resuscitation bag 12 and compresses or constrains the internal volume of air/oxygen contained therein as shown in FIG. 6C.

Disposed or defined at a series of positions along the length of the drawstring 46 are a plurality of demarcations or markers 50 as best seen in FIGS. 6C-6G. According to a known predetermined or premeasured amount, each of the plurality of markers 50 correspond to a different tidal volume contained within the resuscitation bag 12 when that particular marker 50 has been pulled through the spring stop 48. Each marker 50 is denoted by a number, character, or is color coded so as to easily indicate to the user which marker 50 applies to which weight classification of the patient. The user continues to pull the drawstring 46 in the distal direction until the resuscitation bag 12 has been compressed to a desired degree. For example, if the patient is a large adult, the user pulls the distal end of the drawstring 46 to compress the resuscitation bag 12 until the first of the plurality of markers 50 becomes visible after being pulled through the spring stop 48 as seen in FIG. 6C. If the patient is a smaller adult or child, the user instead distally pulls the free end of the drawstring 46 as seen in FIG. 6D until the second or third marker 50 disposed on the drawstring 46 becomes visible after being pulled through the spring stop 48 as best seen in FIG. 6E. If the patient is an infant or exceptionally small, the user may selectively pull the free end of the drawstring 46 until the fourth or final maker 50 disposed on the drawstring 46 becomes visible shown in FIGS. 6F and 6G. At whatever marker 50 the free end of the drawstring 46 is pulled to, the drawstring 46 is then temporarily fixed at that position by the user releasing their grip on the spring stop 48 which automatically pinches or clamps down on the portion of the drawstring 46 disposed therein, thereby preventing any further relative movement between the spring stop 48 and the drawstring 46.

After the drawstring 46 has been properly adjusted and the face mask has been appropriately placed, the resuscitation bag 12 is ready for use by applying a number compressions followed by a corresponding number of release intakes or intake strokes. To perform a compression, the user squeezes or compresses the flexible surface of the resuscitation bag 12 which pushes the air/oxygen disposed in the volume 28 out through the outlet 24 and then subsequently to the patient. After a compression has been completed, the user then relaxes their grip on the resuscitation bag 12 which begins an intake stroke. After each time the resuscitation bag 18 is released, the surface of the bag 12 is resilient enough so that the bag 12 returns or attempts to return to its original shape which draws in new air/oxygen through the valving disposed in the outlet 24 and once again fills the volume 28. Because the drawstring 46 in effect reduces the amount that the internal volume 28 of the resuscitation bag 12 may be re-inflated, the more the drawstring 46 is pulled through the resuscitation bag 12, the less air/oxygen there will be which is capable of being delivered to the patient through compression of the resuscitation bag 12. Conversely, if the drawstring 46 is not pulled distally through the resuscitation bag 12, the larger the volume of air the resuscitation bag 12 will expel with each compression. The spring stop 48 sufficiently fixes or holds the drawstring 46 in place through the resuscitation bag 12 so that even if the user completely releases the resuscitation bag 12, the spring stop 48 will not allow the resuscitation bag 12 to return to its original, full shape and therefore will not affect the volume of air/oxygen being delivered to the patient. To release the drawstring 46, the user actuates the spring stop 48 which allows the drawstring 46 to move in the proximal direction, thereby allowing the resuscitation bag 12 to freely expand back to a new desired volume or even to its original starting volume.

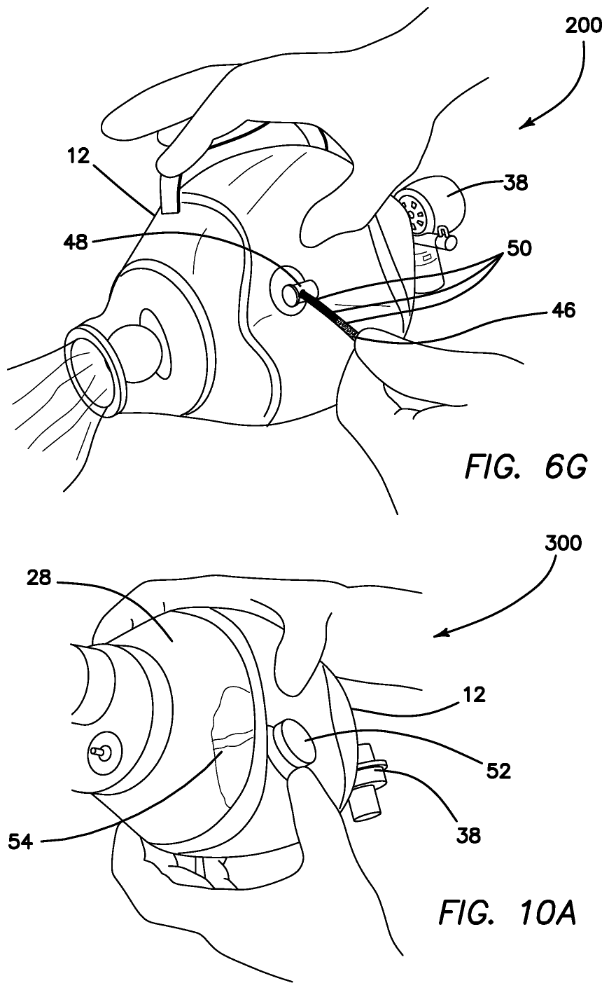

Yet another alternative embodiment of the apparatus 300 may be seen by turning to FIGS. 7A-10G. Here, the resuscitation bag 12 comprises a ratchet 52 coupled to a wire 54 which is defined or disposed through the internal diameter of the resuscitation bag 12. The ratchet 52 is preferably comprised of plastic or plastic composites while the wire 54 is preferably comprised of rope, flexible metal, fabric, plastic, or string. One end of the wire 54 is anchored or coupled in or to a first side of the ratchet 52 disposed on a substantially top portion of the resuscitation bag 12 with the remaining opposing end of the wire being disposed through the internal volume 28 of the resuscitation bag 12 and threaded through a hole or aperture on the opposing surface or wall of the resuscitation bag 12. The wire 54 is then threaded through an adjacent hole or aperture along the opposing surface and then disposed back through the internal volume 28 and coupled in or to a second side of the ratchet 52, the second side of the ratchet 52 being disposed on an opposite side of the ratchet 52 relative to the first side.

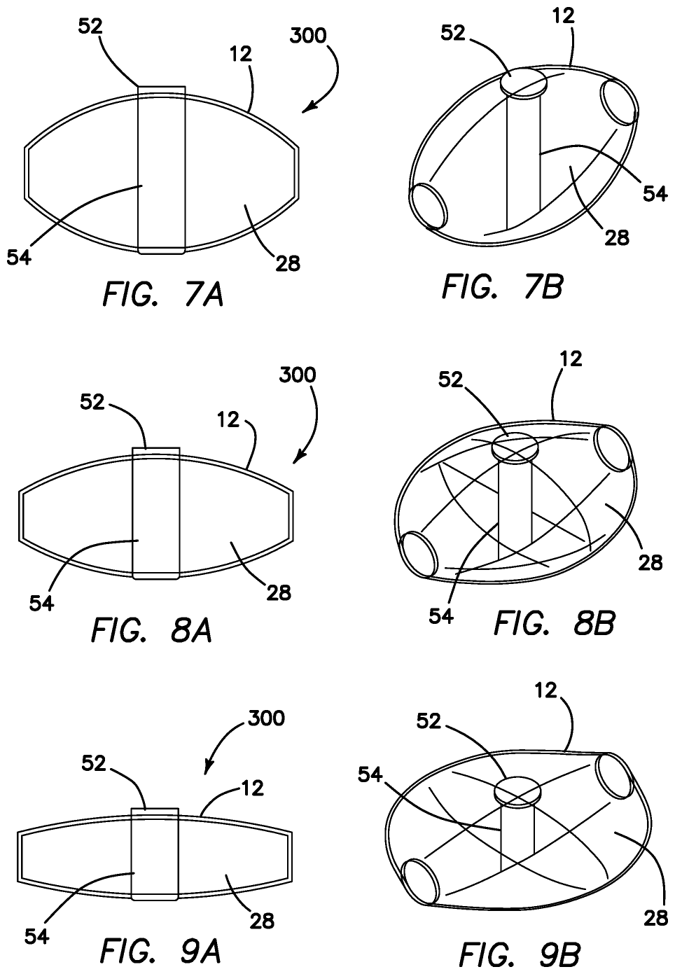

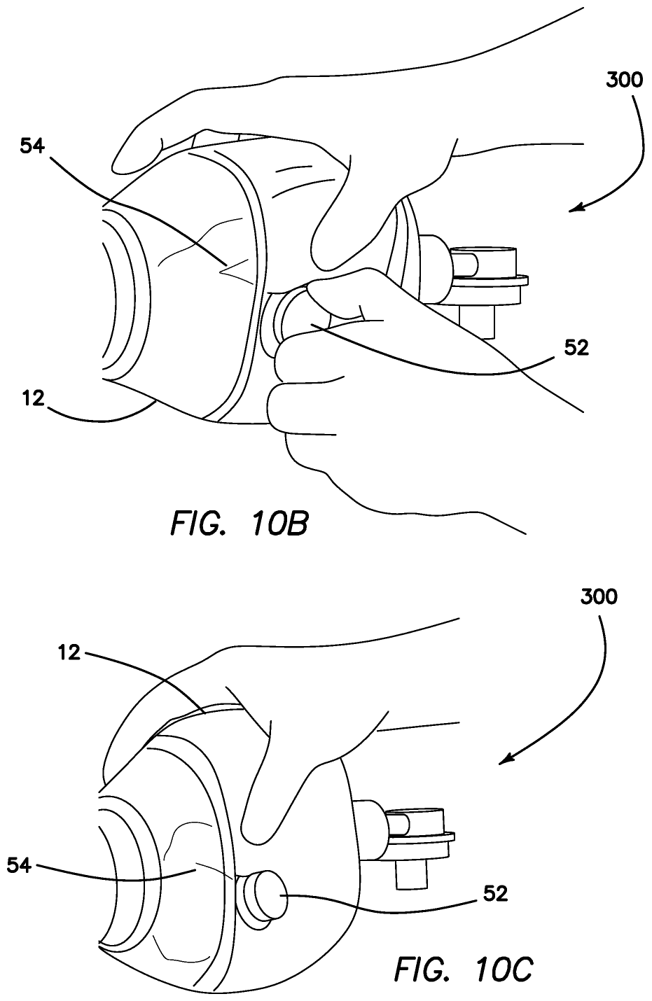

To use the alternative embodiment of apparatus 300, a user approaches a patient requiring ventilation and makes an estimate as to their age and/or weight. The user then adjusts the ratchet 42 which is already disposed through the center of the internal volume 28 of the resuscitation bag 12. The user adjusts the ratchet 52 by rotating it in a first direction which draws or pulls one or both ends of the wire 54 into the housing or body of the ratchet 52 itself as seen in FIGS. 8A and 9A. Because the wire 54 is disposed over a portion or segment of the opposing surface of the resuscitation bag 12, pulling or drawing in the wire 54 deforms the surface of the resuscitation bag 12 and compresses the internal volume 28 of air/oxygen contained therein as shown in FIGS. 8B and 9B.

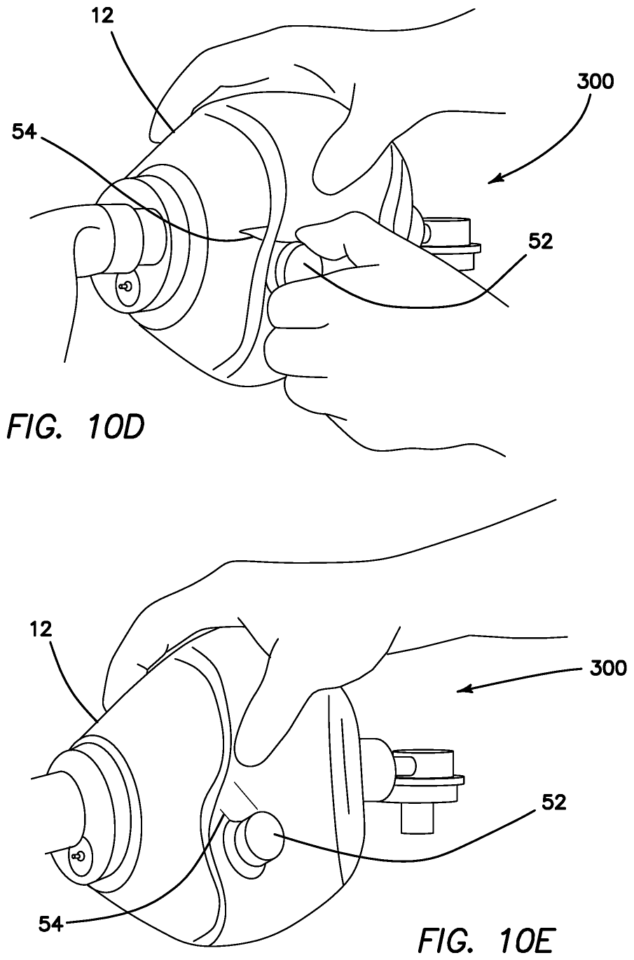

As the ratchet 52 is rotated, it emits a “click” or other audible sound at regular, predetermined intervals. According to a known predetermined or premeasured amount, each time the ratchet 52 “clicks”, a specified length of wire 54 is retracted into the ratchet 52 thereby indicating that a corresponding degree of a tidal volume contained within the resuscitation bag 12 has been achieved. The ratchet 52 may further comprise a display or counter which displays a number, character, or a color so as to easily indicate to the user what level of compression the resuscitation bag 12 is currently adjusted to. The user continues to rotate the ratchet 52 and further pull in the wire 54 until the resuscitation bag 12 has been compressed to a desired degree. For example, if the patient is a large adult, the user rotates the ratchet 52 until two distinct clicks are heard to compress the resuscitation bag 12 as seen in FIGS. 8A, 8B, and 10A. If the patient is a smaller adult or child, the user will continue to rotate the ratchet 52 an additional two or three times as seen in FIG. 10B thereby drawing in an additional length of the wire 54 until the resuscitation bag 12 is further compressed as seen in FIGS. 9A, 9B, and 10C. If the patient is a child or young teen, the ratchet 52 is further rotated as seen in FIG. 10D which results in a further compressed resuscitation bag 12 as best seen in FIG. 10E When treating an infant, the user may selectively rotate the ratchet 52 even further as seen in FIG. 10F to produce a maximally compressed resuscitation bag 12 shown in FIG. 10G. At whatever degree the ratchet 52 is rotated to, the wire 46 is then temporarily fixed at that position by the ratchet 52 itself which automatically pinches, clamps, or otherwise maintains the portion of the wire 54 disposed therein in a fixed position.

After the ratchet 52 has been properly adjusted and the face mask has been appropriately placed, the resuscitation bag 12 is ready for use by applying a number compressions followed by a corresponding number of release intakes or intake strokes. To perform a compression, the user squeezes or compresses the flexible surface of the resuscitation bag 12 as best seen in FIGS. 10C, 10E and 10G which pushes the air/oxygen disposed in the volume 28 out through the outlet 24 and then subsequently to the patient. After a compression stroke has been completed, the user then relaxes their grip on the resuscitation bag 12 which begins an intake stroke. After each time the resuscitation bag 18 is released, the surface of the bag 12 is resilient enough so that the bag 12 returns or attempts to return to its original shape which draws in new air/oxygen through the valving disposed in the outlet 24 and once again fills the volume 28. However because the ratchet 52 and wire 54 in effect reduce the amount that the internal volume 28 of the resuscitation bag 12 may be re-inflated, the more the wire 54 is pulled through the resuscitation bag 12 the less air/oxygen there will be which is capable of being delivered to the patient through compression of the resuscitation bag 12. Conversely, if the wire 54 is not retracted through the resuscitation bag 12 by the ratchet 52, the larger the volume of air the resuscitation bag 12 will expel with each compression stroke. The ratchet 52 sufficiently fixes or holds the wire 54 in place through the resuscitation bag 12 so that, even if the user completely releases the resuscitation bag 12, the ratchet 52 will not allow the resuscitation bag 12 to return to its original, full shape and therefore will not affect the volume of air/oxygen being delivered to the patient. To release the ratchet 52 and lengthen the wire 54, the user actuates the ratchet 52 in an opposing direction which allows the wire 54 to extend or move out of the ratchet 52, thereby allowing the resuscitation bag 12 to freely expand back to a new desired volume or even to its original starting volume.

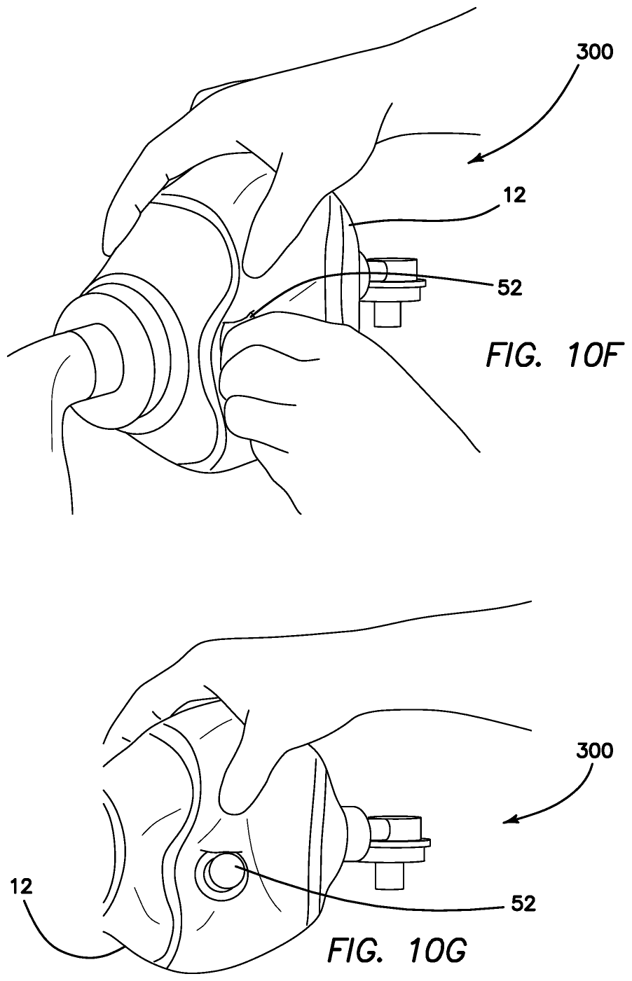

An alternative embodiment of the apparatus 400 may be seen by turning to FIGS. 11A and 11B. Here, the resuscitation bag 12 comprises a plurality of ribs 60 which are wrapped or spiraled around or embedded into the outer surface of the resuscitation bag 12. The ribs 60 are preferably comprised of a rigid or semi-rigid material such as metal, metal alloys, or the like, specifically with a first end of each of the ribs 60 coupled to an adjustable dial 56, and a second end of the ribs 60 coupled to a stationary base 64. The ribs 60 also form a pleated surface 66 as seen in FIG. 11B, thereby allowing the diameter of the resuscitation bag 12 to reduce or contract when the dial 56 is manipulated as discussed further below. A telescoping connecting rod 62 is coupled to the dial 56 and the base 64 so that it is disposed longitudinally through the internal volume 28 of the resuscitation bag 12. Because the plurality of ribs 60 are spiraled around the resuscitation bag 12, each rib 60 is coupled to the dial 56 at a different circumferential position relative to the circumferential position each rib 60 is coupled to at the base 54. In other words, because each rib 60 comprises at least one angled portion, the alignment between the dial 56 and the base 54 for each rib 60 is offset, or asymmetrical.

To use the alternative embodiment of apparatus 400, a user approaches a patient requiring ventilation and makes an estimate as to their age and/or weight. The user then rotates or actuates the dial 56 in a first direction which begins to move the first end of each of the plurality of ribs 60 in the same corresponding direction. Because the second end of each of the plurality of ribs 60 are coupled to the stationary base 64, continual rotation of the dial 56 causes, the ribs 60 to begin to deform and move closer together, thereby causing the pleated surface 66 of the resuscitation bag 12 to likewise deform and compress the internal volume 28 within the resuscitation bag 12. As the diameter of the resuscitation bag 12 is decreased or contracted, the overall length of the resuscitation bag 12 inherently increases which causes the connecting rod 62 to likewise expand in response. Disposed or defined at a series of radial positions around the dial 56 are a plurality of demarcations or markers. According to a known predetermined amount, each of the plurality of markers correspond to a different tidal volume contained within the resuscitation bag 12 when the dial 56 is rotated by that amount. Each marker is denoted by a number, character, or is color coded so as to easily indicate to the user which marker applies to which weight classification of the patient. The user continues to rotate the dial 56 in the first direction until the resuscitation bag 12 has been compressed to a desired degree. For example, if the patient is a large adult, the user rotates the dial 56 to compress the resuscitation bag 12 until a first marker is reached on the dial 56. If the patient is a smaller adult or child, the user instead continues to rotate the dial 56 and bring the ribs 60 together until a second or third marker disposed on the dial 56 is reached. If the patient is an infant or exceptionally small, the user may selectively rotate the dial 56 until reaching the fourth or final maker disposed on the dial 56. At whatever position the dial 56 is rotated or actuated to, the dial 56 is then temporarily fixed at that position by the interaction between the outer surface of the resuscitation bag 12 and the dial 56 itself.

After the dial 56 has been properly adjusted and the face mask has been appropriately placed, the resuscitation bag 12 is ready for use by applying a number compressions followed by a corresponding number of release intakes or intake strokes. To perform a compression stroke, the user squeezes or compresses the flexible surface of the resuscitation bag 12 which pushes the air/oxygen disposed in the volume 28 out through the outlet 24 and then subsequently to the patient. After a compression has been completed, the user then relaxes their grip on the resuscitation bag 12 which begins an intake stroke. After each time the resuscitation bag 18 is released, the surface of the bag 12 is resilient enough so that the bag 12 returns to its original shape which draws in new air/oxygen through the valving 58 and once again fills the volume 28. Because the dial 56 and ribs 60 in effect reduce the amount that the internal volume 28 of the resuscitation bag 12 may be re-inflated, the more the ribs 60 deform the resuscitation bag 12, the less air/oxygen there will be which is capable of being delivered to the patient through compression of the resuscitation bag 12. Conversely, if the ribs 60 remain lose or in a more relaxed state, the larger the volume of air the resuscitation bag 12 will expel with each compression. The ribs 60 themselves are resilient enough around the resuscitation bag 12 so that even if the user completely releases the resuscitation bag 12, the ribs 60 will not expand beyond the setting set by the dial 56 and allow the resuscitation bag 12 to return to its original shape thereby affecting the volume of air/oxygen being delivered to the patient. To expand the ribs 60, the user rotates the dial 56 in an opposing direction which relaxes the ribs 60 and allows them to bend back into their original shape, allowing the pleated surface 66 of the resuscitation bag 12 to freely expand back to a new desired volume or even its original starting volume. As the resuscitation bag 12 expands, the diameter of the resuscitation bag 12 increases which shortens or contracts the length the bag 12 and causes the connecting rod 60 to telescopically contract into a shorter length.

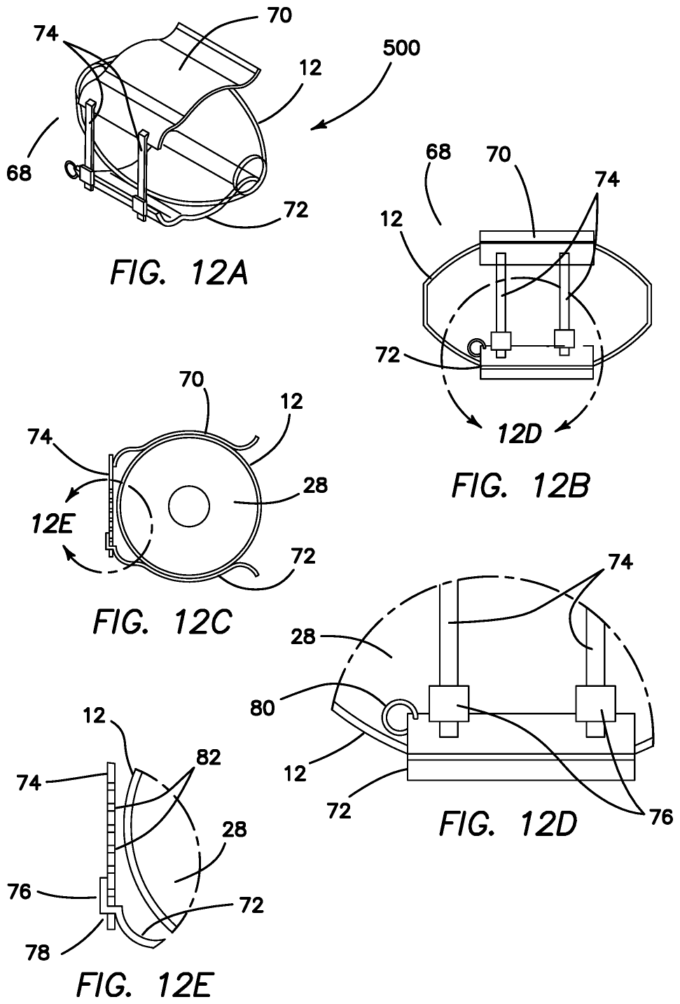

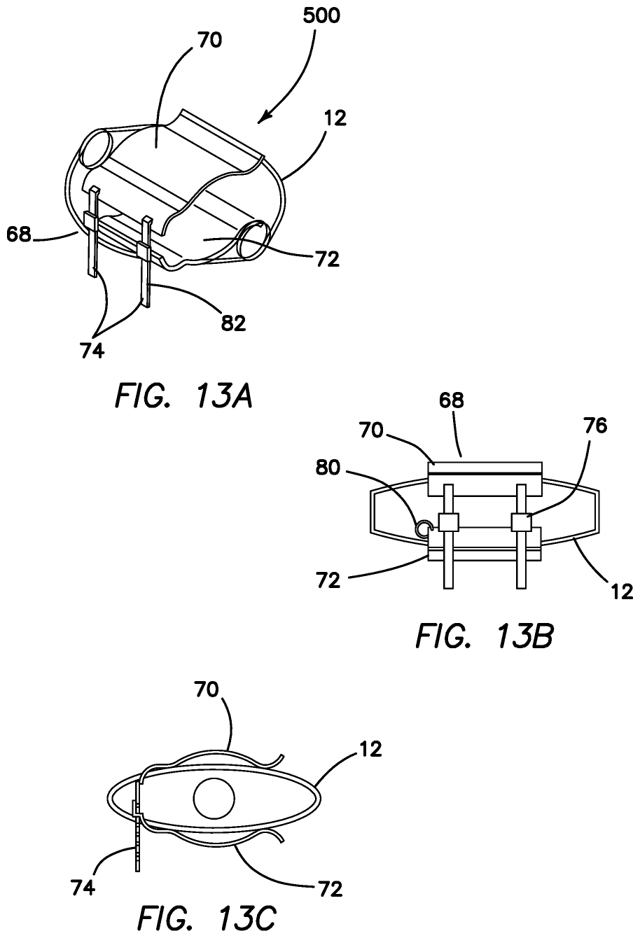

An alternative embodiment of the apparatus 500 may be seen by turning to FIGS. 12A-13C. Here, the resuscitation bag 12 comprises a clamp 68 which encompasses or is disposed around the outer circumference of the resuscitation bag 12. The clamp 68 is preferably comprised of an upper plate 70 and a lower plate 72, specifically with the upper plate 70 disposed over a first portion of the resuscitation bag 12 while the lower plate 72 is disposed over second portion of the resuscitation bag 12, the second portion being diametrically disposed on the opposing side of the resuscitation bag 12 relative to the first portion. Both the upper plate 70 and lower plate 70 comprise matching contoured shapes to substantially accommodate the cylindrical shape of the resuscitation bag 12. The upper plate 70 and lower plate 72 are coupled to a common pair of rails 74 which are orientated parallel to each other and perpendicular relative to the longitudinal axis of the resuscitation bag 12. Each rail 74 comprises a plurality of adjustment holes 82 defined along the height thereof. The lower plate 72 is specifically coupled to the pair of rails 74 via a corresponding pair of apertures 76 which accommodate the distal ends of the rails 74 there through. The lower plate 72 further comprises a pin hole 78 defined horizontally through each of the apertures 76 and a removable adjustment pin 80 which may be disposed therein. As best seen in the magnified view of FIG. 12D, the adjustment pin 80 is removably disposed or threaded through both the pin holes 78 defined through each of the apertures 76 and the adjustment holes 82 defined through each of the rails 74 which correspond to the relative position of the lower plate 72 as is discussed in further detail below. The clamp 68 is preferably coupled to or disposed about the resuscitation bag 12 before treatment of the patient begins, however the clamp 68 may also be initially detached and held separately until the user is ready to begin treatment.

To use the alternative embodiment of apparatus 500, a user approaches a patient requiring ventilation and makes an estimate as to their age and/or weight. The user then adjusts the clamp 68 which is already preferably disposed around the external circumference of the internal volume 28 of the resuscitation bag 12. The user adjusts the clamp 68 by squeezing or compressing the upper plate 70 and the lower plate 72 together which moves the lower plate 72 vertically upward along the rails 74 and closer to the upper plate 70. As the lower plate 72 moves upward along the rails 74, the apertures 76 of the lower plate 72 allow the rails 74 to pass there through. As the user continues to compress the clamp 68, both the upper plate 70 and the lower plate 72 press against the outside surface of the resuscitation bag 12 and begin to deform its surface, thus compressing the internal volume 28 within the resuscitation bag 12. Disposed or defined at or next to the plurality of adjustment holes 82 defined in the rails 74 are a plurality of demarcations or markers. According to a known predetermined amount, each of the plurality of markers correspond to a different adjustment, hole 82 and to a specific tidal volume contained within the resuscitation bag 12 when the lower plate 72 is brought to the adjustment holes 82 that correspond to that particular marker. Each marker is denoted by a number, character, or is color coded so as to easily indicate to the user which marker applies to which weight classification of the patient. The user continues to compress the clamp 68 until the resuscitation bag 12 has been compressed to the desired degree. For example, if the patient is a large adult, the user squeezes the clamp 68 to compress the resuscitation bag 12 until the lower plate 72 is brought into alignment with an adjustment hole 82 defined in each rail 74 which correspond to a first marker. The lower plate 72 is then locked into position by disposing or threading the adjustment pin 80 horizontally through the adjustment holes 82 and the pin holes 78 so that the lower plate 72 remains at the position denoted by the first marker. If the patient is a smaller adult or child, the user instead squeezes the clamp 68 until the lower plate 72 is aligned to a second or third marker disposed on the rails 74 and then locked into, position via the adjustment pin 80. If the patient is an infant or exceptionally small, the user squeezes the clamp 68 until reaching a fourth or final maker disposed on the rails 74 as seen in FIGS. 13A-13C. At whatever marker the lower plate 72 is brought to or aligned with, the lower plate 72 is then temporarily fixed at that position by the user inserting the adjustment pin 80 as discussed above.

In a related embodiment, the upper plate 70 and/or the lower plate 72 are rotatable relative to the vertical rails 74. In this embodiment, the resuscitation bag 12 is compressed by rotating the lower plate 72 towards the upper plate 70 until the desired level of compression is reached, at which point the lower point 72 is locked into position by insertion of the adjustment pin 80.

After the clamp 68 has been properly adjusted and the face mask has been appropriately placed, the resuscitation bag 12 is ready for use by applying a number compressions followed by a corresponding number of release intakes or intake strokes. To perform a compression stroke, the user squeezes or compresses the flexible surface of the resuscitation bag 12 which pushes the air/oxygen disposed in the volume 28 out through the outlet 24 and then subsequently to the patient. After a compression stroke has been completed, the user then relaxes their grip on the resuscitation bag 12 which begins an intake stroke. After each time the resuscitation bag 18 is released, the surface of the bag 12 is resilient enough so that the bag 12 returns to its original shape which draws in new air/oxygen through the valving disposed in the outlet 24 and once again fills the volume 28. Because the clamp 68 in effect reduces the amount that the internal volume 28 of the resuscitation bag 12 may be re-inflated, the more the clamp 68 is actuated about the resuscitation bag 12, the less air/oxygen there will be which is capable of being delivered to the patient through compression of the resuscitation bag 12. Conversely, if the damp 68 is in a more expanded state as seen in FIGS. 12A-12C, the larger the volume of air the resuscitation bag 12 will expel with each compression. The damp 68 is sufficiently locked or fixed into place around the resuscitation bag 12 via the insertion of the adjustment pin 80 so that even if the user completely releases the resuscitation bag 12, the clamp 68 will not allow the resuscitation bag 12 to return to its original shape and therefore will not affect the volume of air/oxygen being delivered to the patient. To release the clamp 68, the user removes or pulls out the adjustment pin 80 from the rails 74 and the apertures 76 of the lower plate 70, thereby allowing the upper plate 70 and the lower plate 72 to separate from each other as the resuscitation bag 12 freely expands back to a new desired volume or even its original starting volume.

Many alterations and modifications may be made by those having ordinary skill in the art without departing from the spirit and scope of the embodiments. Therefore, it must be understood that the illustrated embodiment has been set forth only for the purposes of example and that it should not be taken as limiting the embodiments as defined by the following embodiments and its various embodiments.

Therefore, it must be understood that the illustrated embodiment has been set forth only for the purposes of example and that it should not be taken as limiting the embodiments as defined by the following claims. For example, notwithstanding the fact that the elements of a claim are set forth below in a certain combination, it must be expressly understood that the embodiments includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the embodiments is explicitly contemplated as within the scope of the embodiments.

The words used in this specification to describe the various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself. [98] The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the embodiments.

We claim:

1. An apparatus for providing a selected tidal breath to a patient comprising:
a resuscitation bag comprising a first volume;
means for reducing the first volume of the resuscitation bag to obtain a second volume; and
means for maintaining the second volume of the resuscitation bag during a series of compressions of the resuscitation bag,
wherein the second volume of the resuscitation bag corresponds to the selected tidal breath.

2. The apparatus of claim 1 where the means for reducing the first volume of the resuscitation bag to obtain the second volume comprises:
an inflatable core disposed within the resuscitation bag; and
a hand pump coupled to the inflatable core.

3. The apparatus of claim 2 where the means for maintaining the second volume of the resuscitation bag during a series of compressions of the resuscitation bag comprises the inflatable core being comprised of a rigid, non-deformable material.

4. The apparatus of claim 1 where the means for reducing the first volume of the resuscitation bag to obtain the second volume comprises:
an adjustable cage disposed within the resuscitation bag; and
a central arm inserted through an end of the resuscitation bag and coupled to the adjustable cage.

5. The apparatus of claim 4 where the means for maintaining the second volume of the resuscitation bag during, a series of compressions of the resuscitation bag comprises a plurality of rigid hinges disposed in a corresponding plurality of spokes of the adjustable cage.

6. The apparatus of claim 1 where the means for reducing the first volume of the resuscitation bag to obtain the second volume comprises a removable strap disposed around an outer circumference of the resuscitation bag.

7. The apparatus of claim 6 where the means for maintaining the second volume of the resuscitation bag during a series of compressions of the resuscitation bag comprises a segment of coupling material disposed on the strap.

8. The apparatus of claim 1 where the means for reducing the first volume of the resuscitation bag to obtain the second volume comprises a drawstring disposed through the first volume of the resuscitation bag and along a lateral axis of the resuscitation bag.

9. The apparatus of claim 8 where the means for maintaining the second volume of the resuscitation bag during a series of compressions of the resuscitation bag comprises the drawstring being threaded through a spring stop disposed on an outside surface of the resuscitation bag.

10. The apparatus of claim 1 where the means for reducing the first volume of the resuscitation bag to obtain the second volume comprises an adjustable wire disposed between at least two lateral surfaces of the resuscitation bag, wherein at least a segment of the adjustable wire is disposed across a portion of an outside surface of the resuscitation bag.

11. The apparatus of claim 10 where the means for maintaining the second volume of the resuscitation bag during a series of compressions of the resuscitation bag comprises a pair of opposing ends of the adjustable wire coupled to opposing sides of a ratchet, wherein the ratchet is disposed on the outside surface of the resuscitation bag and opposes the portion of the outside surface of the resuscitation bag which comprises the segment of adjustable wire.

12. The apparatus of claim 1 where the means for reducing the first volume of the resuscitation bag to obtain the second volume comprises a plurality of ribs embedded into an outer surface of the resuscitation bag and a telescoping rod disposed through a longitudinal axis of the resuscitation bag.

13. The apparatus of claim 12 where the means for maintaining the second volume of the resuscitation bag during a series of compressions of the resuscitation bag comprises an adjustable dial coupled to the plurality of ribs and to the telescoping rod.

14. The apparatus of claim 1 where the means for reducing the first volume of the resuscitation bag, to obtain the second volume comprises an upper plate and a lower plate, where the lower plate is disposed on an opposing surface of the resuscitation bag relative to the upper plate.

15. The apparatus of claim 14 where the means for maintaining the second volume of the resuscitation bag during a series of compressions of the resuscitation bag comprises an adjustment pin configured to hold the lower plate at a fixed position relative to the upper plate.

16. A method for providing a selected tidal breath to a patient comprising:
providing a resuscitation bag with a first volume of air or oxygen;
selectively altering the first volume of air or oxygen within the resuscitation bag to obtain a second volume of air or oxygen within the resuscitation bag;
compressing the resuscitation bag to expel the second volume of air or oxygen from the resuscitation bag; and
refilling the second volume of the resuscitation bag with a new supply of air or oxygen.

17. The method of claim 16 where selectively altering the first volume of air or oxygen within the resuscitation bag to obtain a second volume of air or oxygen within the resuscitation bag comprises inflating an inflatable core disposed within the first volume of air or oxygen within the resuscitation bag to reduce the first volume of air or oxygen to the second volume of air or oxygen.

18. The method of claim 16 where selectively altering the first volume of air or oxygen within the resuscitation bag to obtain a second volume of air or oxygen within the resuscitation bag comprises expanding a cage disposed within the first volume of air or oxygen within the resuscitation bag to limit the maximum amount the resuscitation bag may be compressed to the second volume of air or oxygen.

19. The method of claim 16 where selectively altering the first volume of air or oxygen within the resuscitation bag to obtain a second volume of air or oxygen within the resuscitation bag comprises tightening a removable strap disposed around the resuscitation bag to deform an outer surface of the resuscitation bag until the second volume of air or oxygen is obtained.

20. The method of claim 16 where selectively altering the first volume of air or oxygen within the resuscitation bag to obtain a second volume of air or oxygen within the resuscitation bag comprises pulling a drawstring disposed through a lateral axis of the resuscitation bag, to deform an outer surface of the resuscitation bag until the second volume of air or oxygen is obtained.

21. The method of claim 16 where selectively altering the first volume of air or oxygen within the resuscitation bag to obtain a second volume of air or oxygen within the resuscitation bag comprises actuating a ratchet coupled to a first lateral side of the resuscitation bag and contracting a wire coupled to a second lateral side of the resuscitation bag to deform an outer surface of the resuscitation bag until the second volume of air or oxygen is obtained, wherein the second lateral side of the resuscitation bag is disposed on an opposing side of the resuscitation bag relative to the first lateral side.

22. The method of claim 16 where selectively altering the first volume of air or oxygen within the resuscitation bag to obtain a second volume of air or oxygen within the resuscitation bag comprises actuating a dial and compressing a plurality of ribs embedded in an outer surface of the resuscitation bag to deform the outer surface of the resuscitation bag until the second volume of air or oxygen is obtained.

23. The method of claim 16 where selectively altering the first volume of air or oxygen within the resuscitation bag to obtain a second volume of air or oxygen within the resuscitation bag comprises:
compressing the resuscitation bag between an upper plate and a lower plate until the second volume of air or oxygen is obtained; and
locking a position of the lower plate relative to the upper plate.
